(19) United States
(12) Patent Application Publication
(10) Pub. No.: US 2024/0407816 A1
(43) Pub. Date: Dec. 12, 2024

(54) Plate Implant

(71) Applicant: Karl Leibinger Asset Management GmbH & Co. KG, Mühlheim an der Donau (DE)

(72) Inventor: Lüder Mosler, Duderstadt (DE)

(21) Appl. No.: 18/697,778

(22) PCT Filed: Oct. 5, 2022
(86) PCT No.: PCT/EP2022/077628

§ 371 Date: Apr. 2, 2024

(30) Foreign Application Priority Data
Oct. 5, 2021 (DE) 202021003115.4

(51) Int. Cl. A61B 17/80

(52) U.S. Cl. CPC A61B 17/8004

(57) ABSTRACT

The present invention relates to a plate implant for bridging a fracture gap for fixing fractures. The plate implant (1a) is provided with a longitudinal axis (LA) in the direction of the largest extent of the plate implant (1a), a plurality of bores (4) which are arranged spaced apart from each other in the direction of the longitudinal axis (LA), and an elastic longitudinal expansion device (2) which is provided at least between two bores (4). The longitudinal expansion device (2) is configured such that 10 the plate implant (1a) has increased expansibility in the direction of the longitudinal axis (LA) but at the same time has high bending stiffness and torsion stiffness.

In order to fix fractures whilst the bone is healing, implants in the form of plates are often used (so-called plate implants or osteosynthesis plates). These flat, generally elongate, plate implants are provided, for example, with a multiplicity of bores for bone screws, by means of which they are screwed to the bone. They are used to stabilize the fracture gap during healing of the bone and are removed, generally by surgery, after an effective healing process.

In practice, it has been observed that when plate implants are provided, bone healing is delayed or incomplete. This is a result of the fact that a certain amount of movement in the bone gap is required in order to stimulate bone healing. Medical literature refers in this respect to axial movements in the range of a few tenths of millimeters whilst simultaneously suppressing shear and twisting movements.

WO 2021/102591 A1 describes a plate implant having notches in the region of the fracture gap which permit a limited amount of mobility. The notches reduce the load-bearing cross-section of the plate implant in the region of the fracture gap to a thin leaf spring, the bending and longitudinal expansion of which is limited by stops. By reason of design, the implant permits limited pivoting of the fixed bones, but no longitudinal-axial movement thereof.

EP 1 221 308 A1 discloses a vertebral column implant having leaf springs which are used to reduce the bending stiffness of the implant. Increased longitudinal expansion of the implant is not supported by the leaf springs.

Therefore, the object of the present invention is to design a plate implant which bridges and stabilizes the fracture gaps but still permits limited movement in the direction of the (bone) longitudinal axis.

The invention provides a plate implant as claimed in claim 1.

The concept forming the basis of the plate implant in accordance with the invention is the provision of an elastic longitudinal expansion device which is provided at least between two bores. The longitudinal expansion device is configured such that the plate implant has increased expansibility in the direction of the longitudinal axis but at the same time has high bending stiffness and torsion stiffness.

This permits a certain amount of longitudinal movement in the fracture gap in order to stimulate healing of the bone.

According to a preferred development, the longitudinal expansion device has an elastic element which is incorporated into the plate implant.

According to a further preferred development, the elastic element is formed by an elastomer insert.

According to a further preferred development, the plurality of bores is provided along the longitudinal axis, wherein the elastic element extends in a first straight portion offset towards the left edge of the plate implant with respect to a first plurality of bores and in parallel with the first bores, and wherein the elastic element extends in a second straight portion offset towards the right edge of the plate implant with respect to a second plurality of bores and in parallel with the second bores and the first portion and the second portion are connected to each other by a third straight portion of the elastic element, said third portion extending between the two bores.

According to a further preferred development, the longitudinal expansion device comprises at least one leaf spring which is formed by one or more notches in the plate implant.

According to a further preferred development, the leaf springs are formed by u-shaped notches in the plate implant which surround a respective bore.

According to a further preferred development, a first plurality of notches is configured such that the u-shape is open towards the left edge of the plate implant, and a second plurality of notches is configured such that the u-shape is open towards the right edge of the plate implant.

According to a further preferred development, the notches overlap laterally.

According to a further preferred development, the leaf springs are configured such that they are formed by respective first, second and third notches, wherein the first and second notches surround a respective bore at least partially in a u-shaped manner, wherein the first u-shape is open towards the left edge of the plate implant and the second u-shape is open towards the right edge of the plate implant, and wherein the third notches are each arranged perpendicular to the longitudinal axis and are arranged between, and spaced apart from, respective pairs of the first and second notches.

According to a further preferred development, the second notches are spaced apart from, and are opposite, the first notches, wherein the third notches are each arranged perpendicular to the longitudinal axis and are arranged between, and spaced apart from, respective pairs of first and second notches such that the leaf springs are formed.

According to a further preferred development, the plate implant has a body region having a substantially constant first width and a head region widening continuously from the first width.

The invention will be explained in more detail hereinunder with the aid of preferred embodiments and with reference to the figures.

### In the Drawings:

FIG. 1 shows a plate implant in accordance with a first embodiment of the present invention;

FIG. 2 shows a plate implant in accordance with a second embodiment of the present invention;

FIG. 3 shows a plate implant in accordance with a third embodiment of the present invention;

FIG. 4 shows a plate implant in accordance with a fourth embodiment of the present invention;

FIG. 5 shows a plate implant in accordance with a fifth embodiment of the present invention;

FIG. 6 shows a plate implant in accordance with a sixth embodiment of the present invention; and

FIG. 7 shows a plate implant in accordance with a seventh embodiment of the present invention.

Hereinunder, like or functionally identical elements are provided with like reference signs.

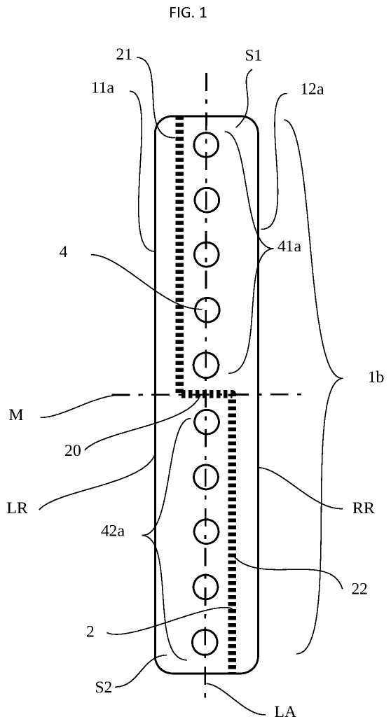

FIG. 1 shows a plate implant in accordance with a first embodiment of the present invention.

The elongate plate implant 1a has a first and a second partial plate 11a, 12a which are connected together via a longitudinal expansion device in the form of an elastic element 2. A plurality of ten bores 4 extends along a central longitudinal axis LA of the plate implant 1a, wherein, in relation to a central transverse axis M of the plate implant, five first bores 41a are arranged on a first side S1 of the central transverse axis M and five second bores 42a are arranged on the other, second side S2 of the central transverse axis M. The elastic element 2 extends in a first straight portion 21 on the first side S1 offset with respect to the first bores 41a towards the left edge LR of the plate implant 1a and in parallel with the first bores 41a, and extends in a second straight portion 22 on the second side S2 offset with respect to the second bores 42a towards the right edge RR of the plate implant 1a and in parallel with the second bores 42a. The first portion 21 and the second portion 22 are connected together by a third straight portion 20 of the elastic element 2 along the central transverse axis M.

During surgery, the fracture gap is arranged along the central transverse axis M and the two partial plates 11a, 12a are attached by means of fixing elements (not shown), e.g. screws, to the two bone fragments separated by the fracture gap. Determining which bores 4 are used for the fixing elements is dependent on the type and course of the fracture gap.

In the event of compression or tension on the fracture gap, the plate implant 1a can be elastically deformed in the direction of the central longitudinal axis LA, without substantially losing any stiffness in the bending and torsion direction.

The elastic element 2 can be configured e.g. as an elastomer insert which does not allow any bending or twisting movements, but does allow longitudinal deformation.

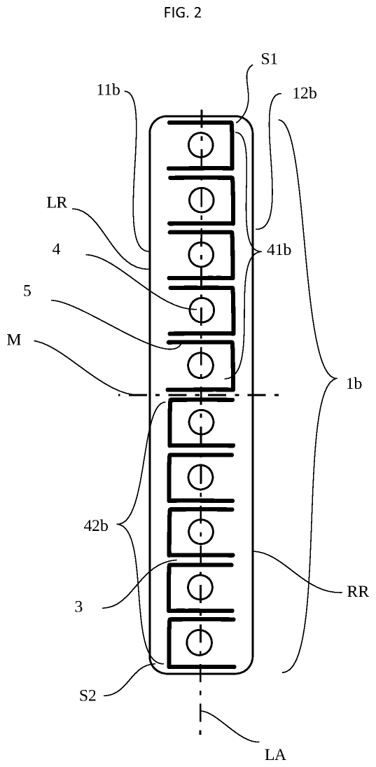

FIG. 2 shows a plate implant in accordance with a second embodiment of the present invention.

The elongate plate implant 1b according to FIG. 2 has a first and a second partial plate 11b, 12b which are arranged on the left and right of the central longitudinal axis LA of the plate implant 1b. The first and second partial plates 11b, 12b have a longitudinal expansion device in the form of a plurality of leaf springs 3.

A plurality of ten bores 4 extends along the central longitudinal axis LA of the plate implant 1b, wherein, in relation to a central transverse axis M of the plate implant, five first bores 41b are arranged on a first side S1 of the central transverse axis M and five second bores 42b are arranged on the other, second side S2 of the central transverse axis M.

The leaf springs 3 extend perpendicularly to the central longitudinal axis LA of the plate implant 1b. They permit elastic deflection in the direction of the central longitudinal axis LA.

The leaf springs 3 are formed by u-shaped notches 5 in the plate implant 1b which surround a respective bore 4. In particular, the notches 5 are provided on the first side S1 of the central transverse axis M such that the u-shape is open towards the left edge LR, and the notches 5 are provided on the second side S2 of the central transverse axis M such that the u-shape is open towards the right edge RR.

The partial plates 11b, 12b and the leaf springs 3 thus form a ladder structure which, owing to the elasticity of the leaf springs, can sheer in a parallelogram-shaped manner and can thus expand in the longitudinal direction.

During surgery, the fracture gap can be arranged along the central transverse axis M and the two partial plates 11b, 12b are attached by means of fixing elements (not shown), e.g. screws, to the two bone fragments separated by the fracture gap. However, in this second embodiment, owing to the symmetry, it is possible for the fracture gap to be arranged offset with respect to the central transverse axis M in the region of a leaf spring. The bores 4 of the partial plates 11b, 12b are then each allocated to the partial plate 11b or the partial plate 12b depending upon the location of the fracture gap.

In the event of compression or tension on the fracture gap, the plate implant 1b can consequently be elastically deformed in the direction of the central longitudinal axis LA, without substantially losing any stiffness in the bending and torsion direction. The sum of the bending stiffnesses of the leaf springs 3 produces the overall stiffness of the plate implant 1b for the desired movement. The largest forces transverse to the leaf spring surface are to be expected in proximity to the location of the fracture gap. It is therefore expedient to make the thickness of the leaf springs 3 thicker or wider in proximity to the location of the fracture gap.

The notches 5 can be produced by methods such as laser beam cutting, water jet cutting or eroding. The cutting width can be suitably selected in order to limit the movement in the direction of the central longitudinal axis LA. Alternatively, the plate implant 1b can also be produced additively, e.g. by laser sintering. In this case, not only the topology of the bone but also the position of the fracture gap can be better taken into account. The material for the plate implant can be the typical corrosion-resistant steels or titanium or titanium alloys.

Particularly in the case of individually producing such a plate implant 1b, it would be alternatively possible to alter the orientation of the leaf springs so that the movement occurring during loading occurs approximately perpendicularly to the surface of the fracture gap. In this respect, the surface of the leaf springs would extend preferably approximately in parallel with the fracture gap.

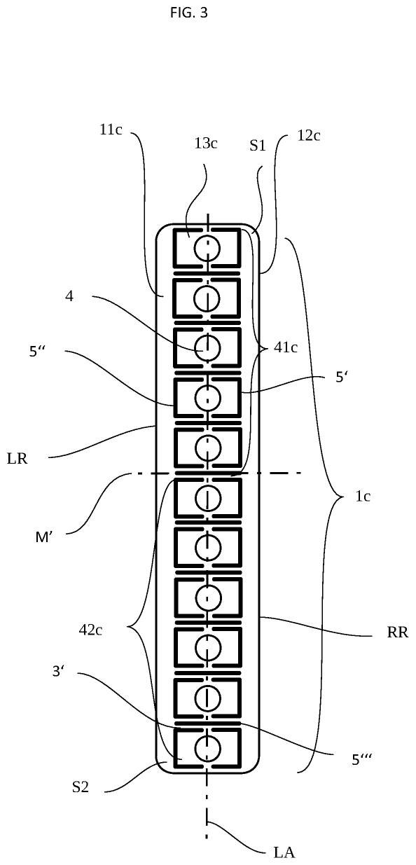

FIG. 3 shows a plate implant in accordance with a third embodiment of the present invention.

FIG. 3 shows an alternative plate implant 1c having a first and second partial plate 11c, 12c and a plurality of third partial plates 13c. As in the previous embodiments, the location of the fracture gap is bridged by the partial plates 11c, 12c, 13c, e.g. in the region of the central transverse axis or, as illustrated, along an axis M′ displaced with respect to the central transverse axis. An advantage in this embodiment is thus likewise that the position of the fracture gap relative to the plate implant 1c is not narrowly defined.

With respect to the displaced axis M′ of the plate implant 1c, five first bores 41c are arranged on a first side S1 of the axis M′ and six second bores 42c are arranged on the other, second side S2 of the axis M′.

The partial plates 13c are connected along the central longitudinal axis LA to leaf springs 3′ as a longitudinal expansion device, which leaf springs are each formed in pairs and hold the other partial plates 11c, 12c. The third partial plates 13c are each provided with a bore 4 along the central longitudinal axis LA.

The leaf springs 3′ are configured such that they are formed by respective first, second and third notches 5′, 5″, 5′ ″, wherein the first notches 5′ delimit the third partial plates 13c on the first partial plate 11c in a u-shaped manner and partially surround the respective bore 4. The second notches 5″ are formed such that they delimit the third partial plates 13c on the second partial plate 12c in a u-shaped manner and partially surround the respective bore 4, wherein they are spaced apart from the first notches 5′.

The third notches 5′″ are each arranged perpendicularly to the central longitudinal axis LA and lie between, and spaced apart from, respective pairs of first and second notches 5′, 5″.

The partial plates 13c hereby have a flexibility in the direction of the longitudinal axis, but are rigidly mounted in the direction of the bore axis and transverse to the longitudinal axis LA.

Not all bores 4 have to be elastically mounted. Such an implant is e.g. suitable for treating fractures in proximity to joints or complex fractures.

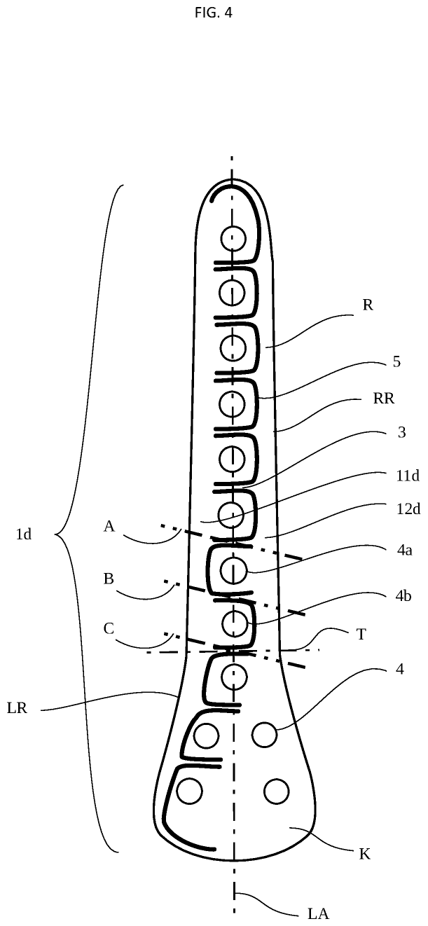

FIG. 4 shows a plate implant in accordance with a fourth embodiment of the present invention.

The elongate plate implant 1d according to FIG. 4 can be used e.g. to treat distal tibia fractures. It has a body region R having a substantially constant first width and a head region K widening continuously from the first width (virtual separating line T).

The partial plates 11d, 12d each extend over the entire length of the plate implant 1d. As in the second embodiment, the first and second partial plates 11d, 12d have a longitudinal expansion device in the form of a plurality of leaf springs 3. A plurality of 13 bores is provided in the plate implant 1d.

In the upper proximal body region R and at the beginning of the lower distal head region K, nine bores 4 are arranged along the central longitudinal axis LA. As the lower distal head region K continues, two bores 4 are allocated to each of the left and right partial plates 11d, 12d and at that location are arranged offset towards the right edge RR or left edge LR.

The leaf springs 3 permit elastic deflection in the direction of the central longitudinal axis LA and are formed by u-shaped notches 5 in the plate implant 1d which surround a respective bore 4 in the body region R along the central longitudinal axis LA. As the upper, proximal head region K continues, two bores 4 are allocated to each of the left and right partial plates 11d, 12d and the leaf springs 3 are offset towards the left edge LR and have an asymmetric u-shape.

In particular, the first six notches 5 and the eighth notch 5 in the body region R are provided such that the u-shape is open towards the left edge LR, whereas the eighth [sic] notch 5 in the body region R is provided such that the u-shape is open towards the right edge RR. The notches 5 in the head region K are also provided such that the u-shape is open towards the right edge RR.

The partial plates 11b, 12b and the leaf springs 3 thus form a ladder structure which, owing to the elasticity of the leaf springs 3, form a longitudinal expansion device which can sheer in a parallelogram-shaped manner.

The alternating arrangement of the leaf springs 3 allows the location of the fracture gap or gaps to be taken into consideration by selecting the screw connections in the bores 4. It is precisely the middle screw connections which are of considerable importance for stabilizing the fracture against bending loads and so it is important to be able to insert screws precisely in this region.

The location of the fracture gap is indicated in the alternative positions A, B or C. If the location of the fracture gap was at A, for example, a screw would be inserted into bore 4a, whereas bore 4b would remain free. If the location of the fracture gap was at B, for example, a screw would not be inserted in either bore 4a or bore 4b. If the location of the fracture gap was at C, for example, a screw would be inserted into bore 4b, whereas bore 4a would remain free.

It can also be seen in FIG. 4 that the leaf springs 3 have, at their ends, soft transitions to the partial plates 11d, 12d. Owing to the reduced notch effect, this improves the fatigue behavior of the plate implant 1d which is important in this respect because the typical materials for implants have a certain notch sensitivity. It can likewise be seen that the resulting width of the partial plates in the area surrounding the location of the fracture gap increases in order to accommodate the larger bending moments which are likely to occur in this region.

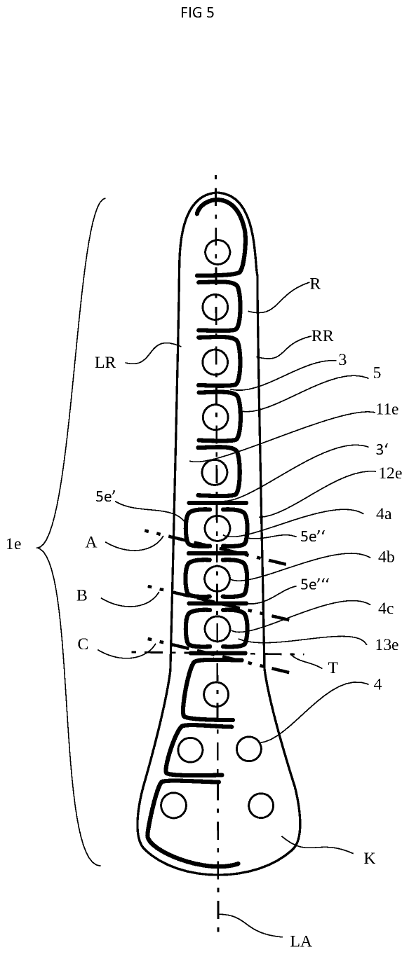

FIG. 5 shows a plate implant according to a fifth embodiment of the present invention, as could be used for example for treating distal tibia fractures.

The plate implant 1e having a first and second partial plate 11e, 12e and a plurality of third partial plates 13e differs from those of the plate implant 1d of the fourth embodiment in that the leaf springs 3′ around the sixth to eighth bore 4 are formed analogously to the leaf springs 3′ of the third embodiment.

In particular, these leaf springs 3′ are configured such that they are formed by respective first, second and third notches 5e′, 5e″, 5e′″, wherein the first notches 5e′ delimit the third partial plates 13e on the first side S1 in a u-shaped manner and partially surround the respective bore 4. The second notches 5e″ are formed such that they delimit the third partial plates 13c on the second side S2 in a u-shaped manner and partially surround the respective bore 4, wherein they are spaced apart from the first notches 5e′. The third notches 5e′″ are each arranged perpendicularly to the central longitudinal axis LA and lie between, and spaced apart from, respective pairs of first and second notches 5e′, 5e″.

The partial plates 13e hereby have a flexibility in the direction of the longitudinal axis, but are rigidly mounted in the direction of the bore axis and transverse to the longitudinal axis LA.

This arrangement likewise allows, irrespective of the position of the location of the fracture gap, screws to be placed in proximity thereto along the positions A, B, C, without thereby limiting the movement in the fracture gap. Therefore, the bores 4a, 4b, 4c can be provided with fixing elements depending upon the fracture gap. This variant is therefore likewise advantageous when fixing fragments.

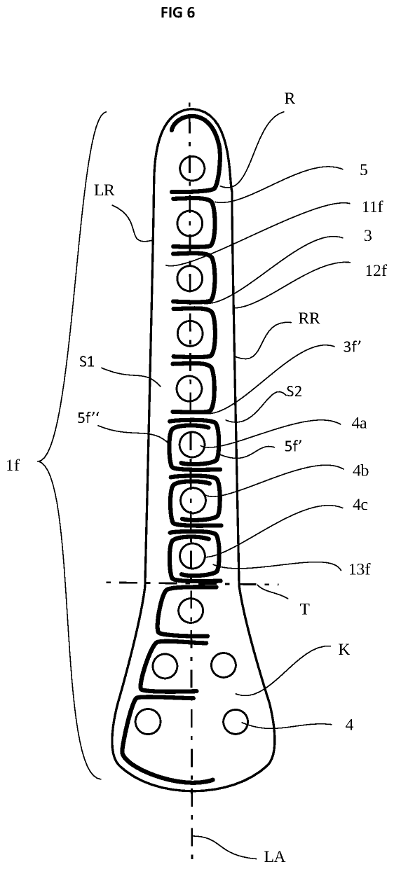

FIG. 6 shows a plate implant in accordance with a sixth embodiment of the present invention.

The plate implant 1f having a first and second partial plate 11f, 12f and a plurality of third partial plates 13f differs from those of the plate implant 1d of the fourth embodiment in that the leaf springs 3f around the sixth to eighth bore 4 are formed such that the mounting of the partial plates 13f is formed with only two notches 5f′, 5f″ of increased length which are spaced apart from each other in an overlapping manner. This produces increased flexibility.

In particular, these leaf springs 3f are configured such that they are formed by respective first and second notches 5f′, 5ef″, wherein the first notches 5f′ delimit the third partial plates 13f on the first side S1 in a u-shaped manner and partially surround the respective bore 4 in an overlapping manner. The second notches 5f″ are formed such that they delimit the third partial plates 13f on the second side S2 in a u-shaped manner and partially surround the respective bore 4, wherein they are spaced apart from the first notches 5f′.

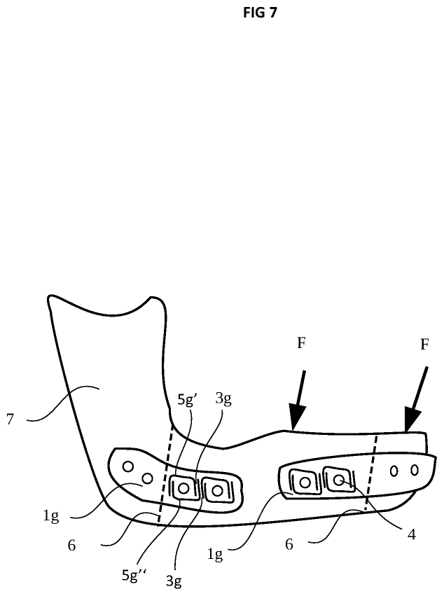

FIG. 7 shows a plate implant according to a seventh embodiment of the present invention used in a reconstruction of a lower jaw 7 from fragments of the fibula.

FIG. 7 shows the use of two plate implants 1g, 1g′ with leaf springs 3g and notches 5g′, 5g″ similar to the leaf springs 3f and notches 5f′, 5f″ according to FIG. 6 in a reconstruction of a lower jaw from fragments of the fibula. Individually configured implant plates 1g, 1g′ which each bridge locations of the respective fracture gap 6 are used.

Instead of rigidly fixing the fragments, the function consists of generating movements and forces by dynamization owing to the existing forces F on portions of the reconstructed lower jaw 7, said movements and forces contributing to compression of the fracture gaps.

This is made possible because the leaf springs 3g are oriented at a slight angle to the location of the fracture gap. A component force is produced which closes the fracture gap.

1. A plate implant for bridging a fracture gap, having:
a longitudinal axis in the direction of a largest extent of the plate implant;
a plurality of bores which are arranged spaced apart from each other in the direction of the longitudinal axis; and
an elastic longitudinal expansion device which is provided at least between two of said plurality of bores;
wherein the longitudinal expansion device is configured such that the plate implant has increased expansibility in the direction of the longitudinal axis but at the same time has high bending stiffness and torsion stiffness.

2. The plate implant for bridging a fracture gap as claimed in claim 1, wherein the longitudinal expansion device has an elastic element which is incorporated into the plate implant.

3. The plate implant for bridging a fracture gap as claimed in claim 1, wherein the elastic element is formed by an elastomer insert.

4. The plate implant for bridging a fracture gap as claimed in claim 2, wherein:
the plurality of bores is provided along the longitudinal axis,
the elastic element extends in a first straight portion offset towards a left edge of the plate implant with respect to a first plurality of said plurality of bores and in parallel with the first plurality of bores,
the elastic element extends in a second straight portion offset towards an opposite right edge of the plate implant with respect to a second plurality of said plurality of bores different from said first plurality of bores and in parallel with the second plurality of bores, and
the first straight portion and the second straight portion are connected to each other by a third straight portion of the elastic element, said third straight portion extending between said two of said plurality of bores.

5. The plate implant for bridging a fracture gap as claimed in claim 1, wherein the longitudinal expansion device comprises at least one leaf spring which is formed by one or more notches in the plate implant.

6. The plate implant for bridging a fracture gap as claimed in claim 5, wherein the at least one leaf spring is formed by u-shaped notches in the plate implant which surround a respective one of said plurality of bores.

7. The plate implant for bridging a fracture gap as claimed in claim 6, wherein a first plurality of said u-shaped notches is configured such that the u-shape is open towards a left edge of the plate implant, and a second plurality of said u-shaped notches is configured such that the u-shape is open towards an opposite right edge of the plate implant.

8. The plate implant for bridging a fracture gap as claimed in claim 6, wherein the notches overlap laterally.

9. The plate implant for bridging a fracture gap as claimed in claim 5, wherein the at least one leaf spring is formed by respective first, second and third notches, wherein the first and second notches surround a respective one of said plurality of bores at least partially in a u-shaped manner, wherein the u-shape of the first notches is open towards a left edge of the plate implant and the u-shape of the second notches is open towards an opposite right edge of the plate implant, and wherein the third notches are each arranged perpendicular to the longitudinal axis and are arranged between, and spaced apart from, respective pairs of the first and second notches.

10. The plate implant for bridging a fracture gap as claimed in claim 5, wherein the at least one leaf spring is formed by respective first, second and third notches, wherein the second notches are spaced apart from, and are opposite, the first notches, and wherein the third notches are each arranged perpendicular to the longitudinal axis and are arranged between, and spaced apart from, respective pairs of first and second notches.

11. The plate implant for bridging a fracture gap as claimed in claim 1, comprising:
a body region extending along said longitudinal axis and having a substantially constant first width; and
a head region at one end of said body region and widening continuously from the first width.

12. The plate implant for bridging a fracture gap as claimed in claim 3, wherein:
the plurality of bores is provided along the longitudinal axis,
the elastic element extends in a first straight portion offset towards a left edge of the plate implant with respect to a first plurality of said plurality of bores and in parallel with the first plurality of bores,
the elastic element extends in a second straight portion offset towards an opposite right edge of the plate implant with respect to a second plurality of said plurality of bores different from said first plurality of bores and in parallel with the second plurality of bores, and
the first straight portion and the second straight portion are connected to each other by a third straight portion of the elastic element, said third straight portion extending between said two of said plurality of bores.
